(19) United States
(12) Patent Application Publication
(10) Pub. No.: US 2012/0022365 A1
(43) Pub. Date: Jan. 26, 2012

(54) Diagnosing Airway Obstructions

(75) Inventor: Perry T. Mansfield, San Diego, CA (US)

(73) Assignee: MANSFIELD ENTERPRISES

(21) Appl. No.: 13/187,391

(22) Filed: Jul. 20, 2011

Related U.S. Application Data
(60) Provisional application No. 61/366,502, filed on Jul. 21, 2010.

(51) Int. Cl. A61B 6/03; A61B 6/00

(52) U.S. Cl. 600/425

(57) ABSTRACT

Methods, devices, and computer program products facilitate characterization of a patient's upper airway including nose, palate, oral cavity, epiglottis, pharynx and larynx. Identification of full or partial obstructions in the upper airway enables the production of a full assessment the patient's airway. A report is produced that details the various contributing factors to the patient's airway obstructions. The comprehensive assessments can be utilized to effectively carry out various surgical and non-surgical procedures for treating sleep apnea.

### CROSS-REFERENCE TO RELATED PATENT APPLICATIONS

This application claims priority from Provisional Application U.S. Application 61/366,502, filed Jul. 21, 2010, incorporated herein by reference in its entirety.

### FIELD OF INVENTION

The present invention relates to the diagnosis and surgical cures of breathing related sleep disorders. More particularly, the present invention relates to characterization of a patient's upper airway to facilitate identification of the specific location of the airway obstructions causing the sleep disorder/apnea.

### BACKGROUND OF THE INVENTION

This section is intended to provide a background or context to the invention that is recited in the claims. The description herein may include concepts that could be pursued, but are not necessarily ones that have been previously conceived or pursued. Therefore, unless otherwise indicated herein, what is described in this section is not prior art to the description and claims in this application and is not admitted to be prior art by inclusion in this section.

Sleep apnea is a common sleep disorder in which an individual stops breathing during the night, sometimes for hundreds of times and for as much as a minute or more. During sleep, the muscle tone of the body ordinarily relaxes, which, in many cases, can cause the soft palate and the tongue to drop backwards causing a temporary closing of the breathing passage way. Obstructive sleep apnea (OSA), which is the most common type of sleep apnea, is characterized by repetitive pauses in breathing during sleep due to the obstruction and/or collapse of the upper airway. A variety of factors contribute to obstructive sleep apnea. Some of these factors include the character of the skeletal and soft tissues supporting the throat, the size of the lower jaw, a large tongue, intrusive tonsils, enlarged adenoids and long floppy soft palate. Excess weight, obesity, hormone changes, certain medications, sleeping pills and aging also increase the risk of sleep apnea. In addition, males have a higher risk of developing sleep apnea.

Obstructive sleep apnea is usually accompanied by a reduction in blood oxygen saturation, and is followed by an arousal and subsequent breathing. Mild occasional sleep apnea, which many people can experience during an upper respiratory infection, may not be a cause for alarm, but chronic severe obstructive sleep apnea requires treatment to prevent low blood oxygen (hypoxemia) and other complications. Obstructive sleep apnea has been further associated with un-refreshed sleep, hypertension, stroke, heart disease, gastric reflux, sexual dysfunction, fatigue, cognitive dysfunction, depression and other conditions.

Sleep apnea is often diagnosed using various levels of polysomnography (PSG). Most PSGs are conducted in a sleep lab. PSG is a comprehensive recording of the biophysiological changes that occur during sleep. PSG can include an array of techniques that are used to monitor many body functions such as brain activity using electroencephalography (EEG), eye movements using electrooculography (EOG), muscle activity or skeletal muscle activation using electromyography (EMG), heart rhythm using electrocardiography (ECG), oxygenation of hemoglobin using pulse oximetry, breathing functions respiratory airflow and respiratory effort indicators.

Once diagnosed, a patient is presented with a variety of treatment options including lifestyle changes that include avoiding alcohol or muscle relaxants, losing weight, and quitting smoking. Other options include sleeping in a particular position, such as sleeping on a side (lateral position) as opposed to sleeping on the back (supine position). Some patients benefit from various kinds of oral/nasal appliances to keep the airway open during sleep. An additional treatment recommendation may also include, a continuous positive airway pressure (CPAP) device may be used to deliver a stream of compressed air to a nasal pillow, nose mask or full-face mask. The air pressure of the CPAP device prevents or limits the airway from obstructing during sleep, thus reducing or eliminating apnea events.

There are also surgical procedures to remove or tighten tissue, widen the airway and/or to correct other obstructions in the airway. While surgery can provide a permanent and effective treatment for obstructive sleep apnea, surgical management has been hindered by the inability to accurately assess the airway anatomy and thereby specify the necessary operation to correct the deformity. It is critical to accurately identify the various obstructions present in the upper airway prior to surgery.

### SUMMARY OF THE INVENTION

The disclosed embodiments relate to methods, devices, and computer program products that facilitate the characterization of a patient's upper airway including nose, palate, oral cavity, epiglottis, pharynx and larynx. The disclosed embodiments allow for the accurate and precise identification of full or partial obstructions in the upper airway that are likely to contribute and/or cause to sleep apnea. On aspect of the disclosed embodiments relates to a method that comprises processing a plurality of images associated with craniofacial features of a patient, where the images having been produced using computed tomography (CT). The method further includes identifying a plurality of landmarks within the processed images and conducting a plurality of measurements in accordance with the identified landmarks to produce one or more parameters. The method also includes producing a comprehensive assessment of the patient's airway in accordance with the parameters.

An of the additional feature of the above method includes producing the comprehensive assessment by comparing the measured parameters to a set of normal values. According to another embodiment, the method further comprises identifying one or more characteristics of the airway using the processed images and producing the comprehensive assessment in accordance with the identified characteristics. In one variation, the characteristic is a volume and shape of the airway and the comprehensive assessment comprises a volume measurement associated with one or more sections of the airway. For example, the one or more sections of the airway are selected from a group of sections of the airway consisting of: a section of the airway with a narrowing, a section of the airway at uvula at its narrowest airway point in a lateral dimension and a section of the airway at base of tongue at its narrowest point in a lateral projection.

According to another embodiment, the above-noted identified characteristic is an area of the airway and the comprehensive assessment comprises an area measurement associated with one or more sections of the airway. The one or more sections of the airway are, for example, be selected from a group of sections of the airway consisting of: a section of the airway with a smallest cross-sectional area, a section of the airway with a narrowing, a section of the airway at uvula, and a section of the airway at base of tongue. In yet another embodiment, the above-noted identified characteristic is associated with nasal fossa, and the comprehensive assessment comprises identified abnormalities within the nasal fossa. For example, the identified abnormalities can be selected from a group of abnormalities consisting of: a septal deviation, a nasal spurring, concha bullosa, innferior turbinate position/swelling, sinusitis, osteomeatal unit occlusion, narrowness of bony nasal structures, prominence of maxillary crest/spine, adenoid swelling and polyps.

According to another embodiment, a plurality of abnormalities within the airway are identified. In yet another embodiment, the above-note method further comprises identifying a specific location of geniotubercle relative to another location selected from a group consisting of: a tooth, mandible, a dental root, and a mental foramen. In still another embodiment, the above-note method further comprises identifying a distance between teeth or a length of a tooth, while in another embodiment, the plurality of images are produced in a single imaging session. In some embodiments, the processing of the plurality of images comprises rendering two- and three-dimensional images of the airway.

In other embodiments, the parameters that are produced according to the above-noted method are selected from a group of parameters consisting of: an SNA angle, an SNB angle, an ANB angle, a PNS-P distance, an MP-H distance, a posterior airway space (PAS) at uvula distance, and a PAS at base of tongue (BOT) distance. Further details regarding these and other parameters are described in the sections that follow. In another embodiment, the landmarks that are identified according to the above-noted method are identified by manually viewing the images. In yet another embodiment, the measurements that are carried out according to the above-noted method are conducted automatically using a computer-implementable algorithm. In still another embodiment, the above-noted method further comprises producing a report comprising a description of one or more abnormalities of the patient's airway. The assessment that is produced in accordance with the above-noted operations provides a reliable diagnosis of sleep apnea.

Another aspect of the disclosed embodiments relates to a device that comprises a processor and a memory comprising processor executable code. The processor executable code, when executed by the processor, configures the device to process a plurality of images associated with craniofacial features of a patient, the images having been produced using computed tomography (CT). The processor executable code, when executed by the processor, further configures the device to identify a plurality of landmarks within the processed images, conduct a plurality of measurements in accordance with the identified landmarks to produce one or more parameters, and produce a comprehensive assessment of the patient's airway in accordance with the parameters.

Another aspect of the disclosed embodiments relates to a computer program product, embodied on a computer-readable medium that comprises computer code for processing a plurality of images associated with craniofacial features of a patient, the images having been produced using computed tomography (CT). The computer program product, embodied on a computer-readable medium further comprises computer code for identifying a plurality of landmarks within the processed images, computer code for conducting a plurality of measurements in accordance with the identified landmarks to produce one or more parameters, and computer code for producing a comprehensive assessment of the patient's airway in accordance with the parameters.

These and other advantages and features of various embodiments of the present invention, together with the organization and manner of operation thereof, will become apparent from the following detailed description when taken in conjunction with the accompanying drawings

### BRIEF DESCRIPTION OF THE DRAWINGS

Embodiments of the invention are described by referring to the attached drawings, in which:

FIG. 1 illustrates the generation of an assessment of airway characteristics in accordance with the disclosed embodiments;

FIG. 2 illustrates various landmarks associated with craniofacial architecture;

FIG. 3 is an image of a sisson nasal angle A (SNA) measurement in accordance with an example embodiment;

FIG. 4 is an image of a sisson nasal angle B (SNB) measurement in accordance with an example embodiment;

FIG. 5 is an image of an ANB angle measurement in accordance with an example embodiment;

FIG. 6 is an image of a posterior nasal spine to the tip of the palate (PNS-P) measurement in accordance with an example embodiment;

FIG. 7 is an image of an mandible plane to hyoid (MP-H) measurement in accordance with an example embodiment;

FIG. 8 is an image of posterior airway space (PAS) measurements at uvula and at the base of tongue in accordance with an example embodiment;

FIG. 9 is an image of an airway area measurement at uvula in accordance with an example embodiment;

FIG. 10 is an image of an airway area measurement at the base of tongue in accordance with an example embodiment;

FIG. 11 is an image of an airway volume measurement in accordance with an example embodiment;

FIG. 12 is an image of the nasal cavity in accordance with an example embodiment;

FIG. 13 is an image of the location of geniotubercle produced in accordance with an example embodiment;

FIG. 14 is an image of teeth distance measurements in relation to the geniotubercle, mandible base, individual dentition, produced in accordance with an example embodiment;

FIG. 15 is an image of teeth length measurements produced in accordance with an example embodiment;

FIG. 16 illustrates an exemplary report produced in accordance with an example embodiment;

FIG. 17 illustrates an exemplary system within which various disclosed embodiments can be implemented;

FIG. 18 illustrates an exemplary device within which various disclosed embodiments can be implemented; and

FIG. 19 is another illustration of various landmarks associated with craniofacial architecture.

### DETAILED DESCRIPTION OF CERTAIN EMBODIMENTS

In the following description, for purposes of explanation and not limitation, details and descriptions are set forth in order to provide a thorough understanding of the disclosed embodiments. However, it will be apparent to those skilled in the art that the disclosed methods, devices and computer program products may be implemented in other embodiments that depart from these details and descriptions.

Additionally, in the subject description, the word “exemplary” is used to mean serving as an example, instance, or illustration. Any embodiment or design described herein as “exemplary” is not necessarily to be construed as preferred or advantageous over other embodiments or designs. Rather, use of the word exemplary is intended to present concepts in a concrete manner.

As noted earlier, obstructive sleep apnea is usually diagnosed by a test called polysomnogram (PSG), which is also commonly known as a sleep study. The study measures multiple body functions while the patient is asleep. Once diagnosed as having sleep apnea, a patient is typically presented with a variety of non-surgical options. However, in many cases, non-surgical methods fail to provide a sustainable or optimal remedy. For example, the use of a continuous positive airway pressure (CPAP) device during sleep can be extremely inconvenient, uncomfortable, embarrassing, and forces the patient to sleep in a different position that may not be practical or sustainable. In a typical course of treatment, a patient is first treated using non-surgical methods and only when non-surgical remedies are unsuccessful, surgical options are considered.

A number of different surgical procedures have been developed to correct specific problems that contribute to obstructive sleep apnea. One set of procedures includes nasal and septal surgeries that are performed to open the nasal breathing passages to allow easier breathing. The obstructive tissue around and within the nostrils can cause restricted breathing. Weak or displaced cartilages, a deviated suptum and excessively narrow nostrils can also impede nasal breathing. In these and other scenarios, external reconstructive surgery (rhinoplasty) may be performed to reduce the blockage of the nasal passage.

Another surgical procedure is uvulo-palato-pharyngoplasty (UPPP). The UPPP shortens and stiffens the soft palate by partial removal of the uvula and reduction of the soft palate. Tonsillectomy is another procedure that involves the removal of tonsils to allow the opening of the blocked upper airway. Genioglossus Advancement (GGA) is another procedure that is designed to treat obstructive sleep apnea. The genioglossus is the primary muscle of the tongue and is attached to a small bony projection on the interior of the lower jaw. During GGA surgery, this small projection is moved forward and the tongue attachment is repositioned anteriorly so that it is less likely to collapse posteriorly and block the airway during sleep. Genioglossus advancement requires precise identification of the geniotubercle. Advancement results in expansion of the posterior airway space increasing airway volume and calbre in the base of tongue region.

In another surgical procedure, called hyoid suspension, the base of the tongue and epiglottis are moved forward to increase the breathing passage. The hyoid bone is a unshaped bone in the neck located just above the thyroid cartilage and has attachments to muscles of the tongue and other soft tissues of the throat. By detaching two tendons on the upper surface of the hyoid bone and some of the muscle on the lower surface, the hyoid can be advanced over the thyroid cartilage and secured in position. Maxillo-mandibular Advancement (MMA) is yet another surgical procedure. In an MMA procedure, the upper jaw (Maxilla) and lower jaw (Mandible) are surgically moved forward. By moving the jaws forward, the soft tissues of the tongue and palate are also advanced, thereby opening the airway in both tongue and palate regions.

Somnoplasty is another surgical procedure which treats sleep apnea by shrinking the soft tissue in the upper airway, and base of tongue. This is accomplished by inserting a small probe into the obstructing tissue. The probe is used to administer controlled heating of the tissue by, for example, applying a low-power RF energy. As a result, coagulative lesions beneath the lining of the targeted areas are created. Over the course of a few weeks, the lesions are naturally resorbed, thus stiffening, contracting and reducing the volume of the tissue thus widening the airway.

It should be noted that the above described exemplary surgical procedures are not intended to provide an exhaustive list of surgical remedies for obstructive sleep apnea. These and other procedures demonstrate the availability of myriad surgical methods that are designed to correct specific airway obstructions that contribute to obstructive sleep apnea. These examples further illustrate that many factors can contribute to the blockage of the upper airway, which must be correctly and precisely identified prior to conducting any surgical procedures.

The disclosed embodiments facilitate comprehensive and accurate identification of the various factors that can contribute to obstructive sleep apnea in a particular patient. The identified factors can then be used to effectively administer the proper surgical and/or non-surgical remedies. According to some embodiments, imaging techniques, such as computed tomography (CT) scans, are used to compute various parameters. The computed parameters, in conjunction with 2- and 3-dimensional images, can then be used to provide a comprehensive analysis of a patient's overall upper airway physiology, which enables accurate identification of various obstructions that contribute to the blockage of the airway.

One technique for analyzing the craniofacial architecture in patients with sleep apnea is cephalometry. Cephalometry utilizes lateral plain X-ray imaging radiographs to gauge the size and spatial relationships of the teeth, jaws and cranium. Comparison of the X-ray images with standard images that correspond to “normal” patients can then, to some extent, reveal the abnormalities in the airway. However, the images that are obtained through cephalometry correspond to a patient that is in the upright rather than supine position. Therefore, such X-ray images often fail to provide an accurate assessment of the patient's airway when patient is asleep. Further, the information obtained via cephalometry only corresponds to two planes and, therefore, fail to provide a complete analysis of the patient's craniofacial architecture.

Images produced through cephalometry also suffer from a lack of reproducibility, since it is often difficult to produce the same x-ray images (i.e., at the same angle and plane) during the course of a patient's treatment, which can last weeks or months. Similarly, it is difficult to consistently and accurately identify the locations of various anatomical points of interest (i.e., landmarks) within such images. As a result, the accuracy of cephalometric measurements and the subsequent analysis can also be adversely affected.

Computed tomography (CT) is another more accurate and reproducible technique for analyzing the three dimensional craniofacial architecture in patients with sleep apnea. CT can assist in providing cross sectional and volume measurements that can yield more accurate information regarding the airway, without the limitations of traditional cephalometry scanning methods. As a result, the CT images provide the cross sectional and volume measurements needed to accurately identify the obstructions in the airway. The disclosed embodiments, provide a turnkey protocol that allows the generation of comprehensive and accurate information that can be used to analyze and assess all obstructions in a patient's airway, assisting in diagnosis, treatment and preoperative planning.

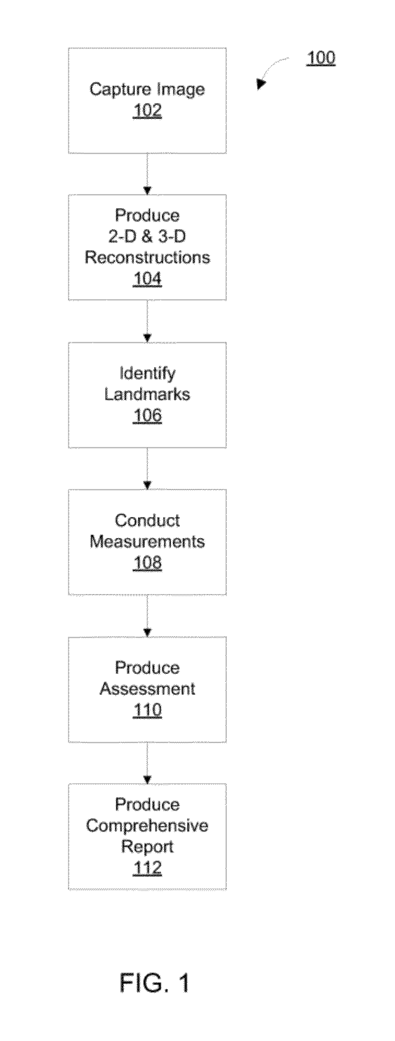

FIG. 1 is a block diagram illustrating a procedure 100 for providing comprehensive assessment of a patient's airway according to an exemplary embodiment. While the block diagram of FIG. 1 is separated into a number of distinct steps in order to facilitate the understanding of the underlying concepts, it is understood that the disclosed processes can include fewer or additional steps. For example, in some embodiments, some of the steps may be combined, while in other embodiments certain steps may be removed. In still other embodiments, additional steps may be added.

Referring back to FIG. 1, in step 102, an imaging technique, such as computed tomography (CT), is used to capture images of the patient's upper airway. In one example, high-resolution CT examinations of the skull base and neck is conducted using a General Electric Lightspeed Ultra 8 slice scanner. Such a CT scanner is configured to produce a plurality of slices (e.g., 64 slices) as part of the imaging procedure. Images may, for example, be obtained from scout anteroposterior and lateral scans that are followed by scans from above the orbits through the bottom of the thyroid cartilage without intravenous contrast. The patient can be instructed not to breathe or swallow during the examination. The scans may also be performed while the patient is in the supine position, with his/her neck in neutral position. This way, the captured images more closely resemble the true characteristics of a patient's upper airway during sleep. In another embodiment, the scans may be conducted while the patient is asleep or is unconscious. For example, a Müller maneuver may be utilized to simulate sleep conditions. In this maneuver, the patient attempts to inhale with his mouth closed and his nostrils plugged, which can lead to a collapse of the airway.

In step 104, various two-dimensional and three-dimensional images are reconstructed. In one example, sagittal and coronal multiplanar reformats are constructed while in another example, multiple three-dimensional reconstructions are produced. The three-dimensional reconstructions may be produced using images that are obtained in step 102 with the aid of a three-dimensional reconstructions software.

In step 106, a plurality of landmarks are identified within the reconstructed images. By the way of example, and not by limitation, such landmarks include soft tissue profile of face, sella turcica (i.e., the saddle-shaped depression in the sphenoid bone at the base of the skull), frontal bone, nasal bone, orbital floor, external auditory meatus, maxilla, upper first molar and upper central incisor, mandible, mandibular symphysis (i.e., a small vertical ridge that marks the fusion of the left and right parts of the mandible), lower first molar, lower central incisor and the like. In some embodiments, the landmarks are identified manually by viewing the images. Such manual identification may also comprise tracing and/or marking the printouts or electronic versions of the images to clearly identify the desired landmarks and prepare the images for subsequent processing steps. In other embodiments, a computerized landmark recognition technique may be used to automatically identify the desired landmarks. For example, image and feature recognition techniques may implemented in software and/or hardware to process the captured and/or reconstructed images in order to automatically identify the desired features and landmarks. These image and feature recognition techniques may include a variety of filtering, scaling and rotation of images, spatial and frequency transformations (e.g., via discrete cosine and/or wavelet transformations), edge detection techniques and other signal and image processing operations that enable the identification of the landmarks. Such image processing techniques may be broken up into several steps that for example, include pre-processing operations such as noise reduction, image sharpening, equalization and the like. The pre-processed images may further processed to effect feature recognition.

The identified landmarks are subsequently used in step 108 to conduct various measurements. These measurements identify a number of different characteristics associated with the upper airway. In particular, the measurements in step 108 may produce the following parameters: SNA, SNB and ANB angles, PNS-P and MP-H distances, posterior airway space (PAS) at uvula, PAS at base of tongue (BOT), area of OP uvula, area of OP BOT, volume of pharynx/larynx, nasal fossa (including septal deviations, spurring, concha bullosa, etc.) location of genio tubercle, teeth distances (e.g., distances from the tip of mandibular canines to the bottom of the mandible and distance between the two roots), teeth lengths (e.g., lengths of both the medial and lateral incisors) and the like. The above noted parameters and characteristics, as well as their significance are further described in sections that follow. It should be noted that the above noted parameters and characteristics are not intended to be provide an exhaustive listing of all parameters or characteristics that can be obtained in accordance with the disclosed embodiments. Therefore, additional or fewer parameters can be obtained and utilized.

In step 110, a comprehensive assessment of the upper airway is produced. This assessment may be conducted by comparing the measured airway characteristics that were obtained in step 108 with “standard” or “normal” measurement results. The standard results, for example, can represent statistical averages of the parameters, as well as standard deviations, that are obtained from a large sample of the population that do not suffer from sleep apnea. Table 1 provides a listing of standard or normal values associated with some of the parameters that are used to assess the airway. The numbers that appear in Table 1 correspond to caucusing population.

The standard values may also differ across populations according to their ethnicity. Therefore, the standard or normal values may be categorized based on ethnicity of the patients. Measurements that significantly deviate from the standard values can identify particular obstructions within the airway.

TABLE 1
Normal Values of Parameters

| Parameter | Normal Value |
|---|---|
| SNA: | 82 ± 2 (degrees) |
| SNB: | 80 ± 2 (degrees) |
| ANB: | 2 (degrees) |
| PNS-P | 35 ± 3 (mm) |
| MP-H | 15 ± 2 (mm) |
| PAS at uvula: | 11 ± 2 (mm) |
| PAS at BOT: | 11 ± 2 (mm) |
| Area of OP at uvula: | Greater than 100 (mm²) |
| Area of OP at BOT: | Greater than 100 (mm²) |
| Volume of pharynx/larynx: | Not available |

In step 112 of FIG. 1, a detailed assessment of the airway, including one or more conclusions as to the cause of airway blockage, is produced in the form of a report. Such a report may be accompanied by a different images and/or measurements that were produced in steps 104 through 108. The report that is generated in step 112 may be in an electronic format, produced as paper copies and/or as film slides.

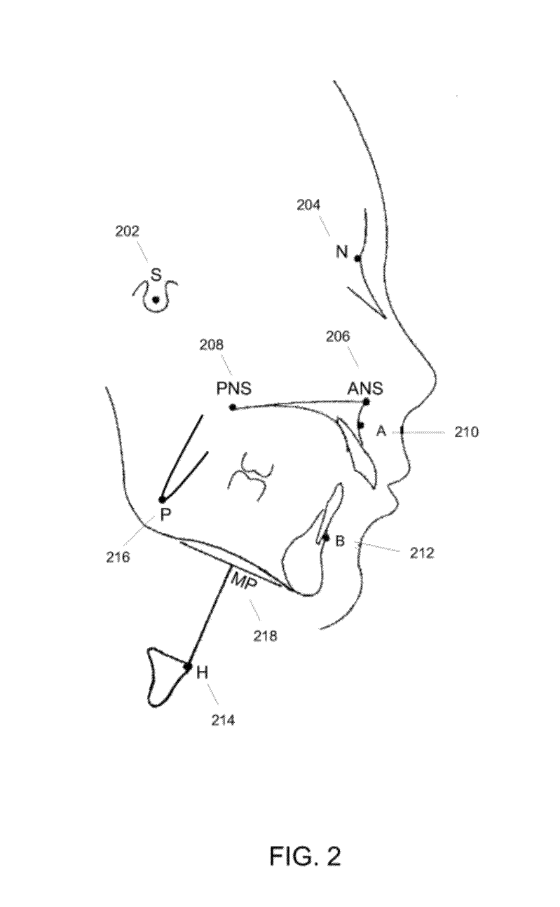

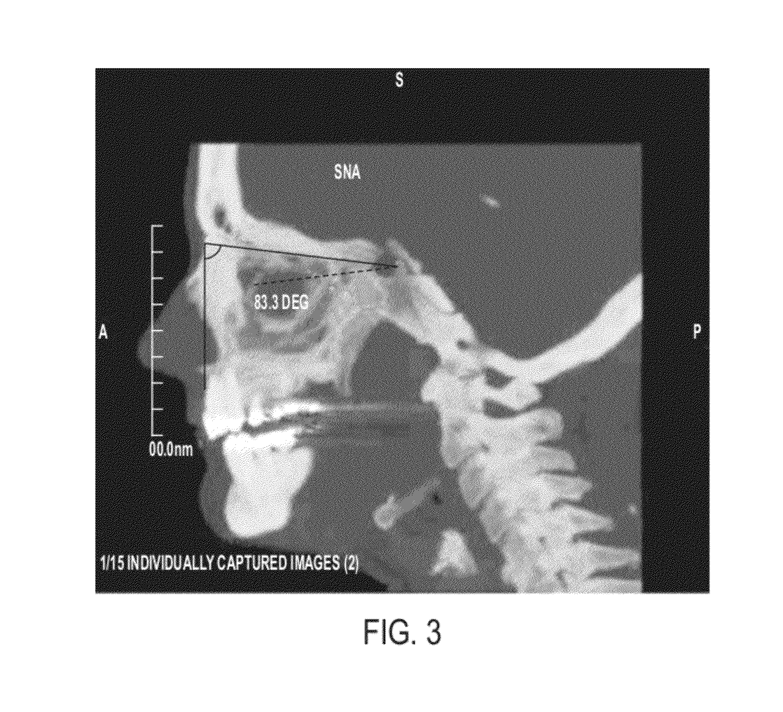

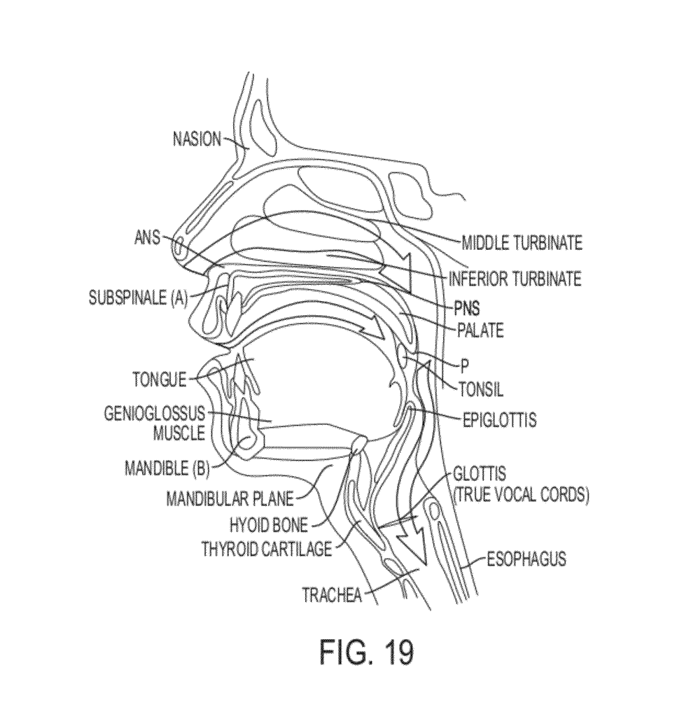

FIG. 2 provides further clarifications as to the locations of certain landmarks and certain measured parameters. FIG. 19 provides a similar diagram as FIG. 2, in which some of the features and points of interest are illustrated with better clarity. With reference to FIG. 2, sella (S) 202 is the mid point of sella turcica, nasion (N) 204, which is the outer most anterior point on fronto-nasal suture, anterior nasal spine (ANS) 206, posterior nasal spine (PNS) 208, point A 210, which is the location of deepest concavity on the anterior profile of maxilla, point B 212, which is the location of the deepest concavity on anterior profile of mandibular symphysis, hyoid (H) 214, which is the location of the hyoid, and point P 216, which is the tip of the soft palate. With the aid of the above identified points, various distances and angles can be measured to characterize the craniofacial architecture of a patient. The disclosed embodiments enable precise, reproducible and accurate measurements of the various parameters associated with the various landmarks and features of interest. For example, SNA represents the angle that is formed by connecting points S 202 to N 204 to A 210. FIG. 3 illustrates an exemplary SNA measurement that has been produced in accordance with the disclosed embodiments. The measured SNA in the exemplary depiction of FIG. 3 is 83.3 degrees, which is within the normal range for this parameter (see Table 1).

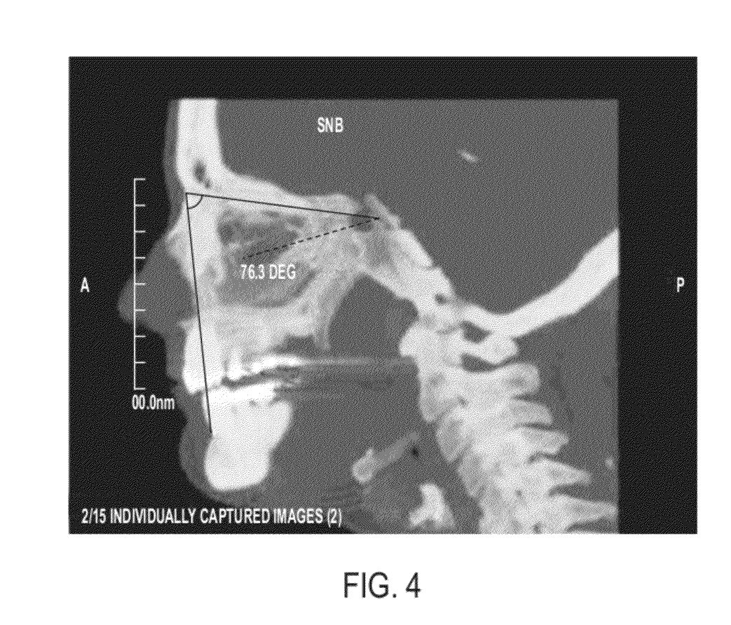

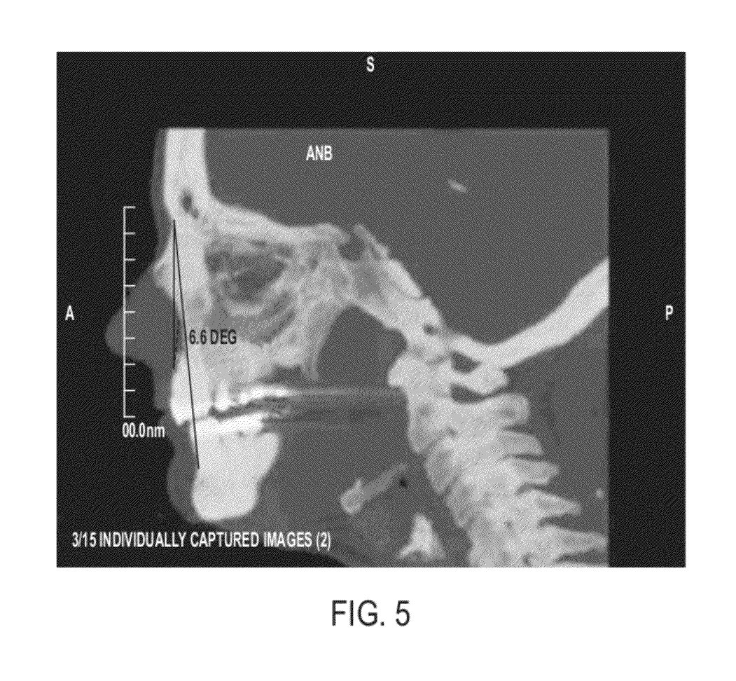

SNB and ANB represent the angles formed by connecting points S 202 to N 204 to B 212, and points A 210 to N 204 to B 212, respectively. These angles indicate the relative position of maxilla/mandible to each other and to the cranial base. FIG. 4 illustrates an exemplary SNB measurement that has been produced in accordance with the disclosed embodiments. The measured SNB in the exemplary depiction of FIG. 4 is 76.3 degrees, which is lower than the normal range for this parameter (see Table 1). FIG. 5 illustrates an exemplary ANB measurement that has been produced in accordance with the disclosed embodiments. The measured ANB in the exemplary depiction of FIG. 5 is 6.6 degrees, which is considerably higher than the normal range for this parameter (see Table 1). The exemplary values of SNB and ANB, which are both outside of the normal range, indicate that the lower jaw is positioned further than normal and may, therefore, be contributing to the narrowing of the airway.

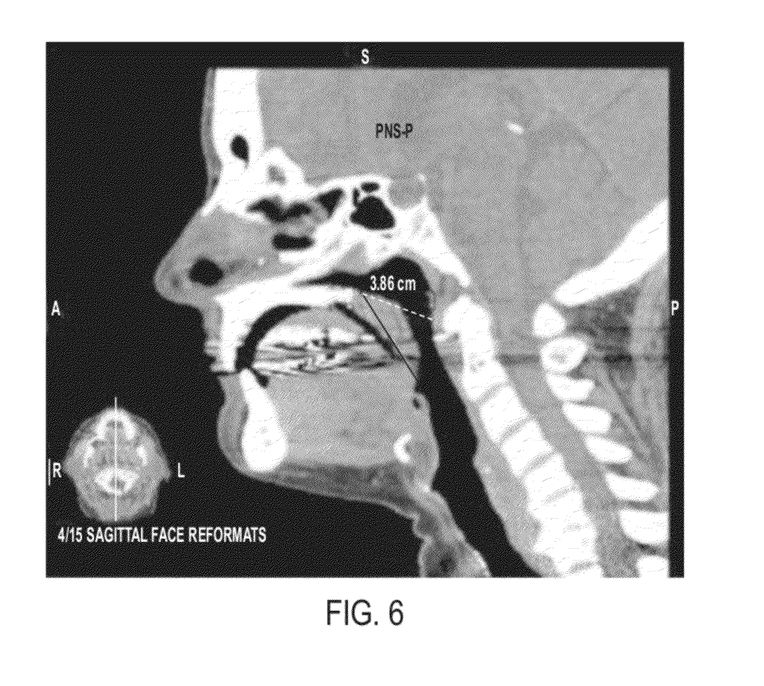

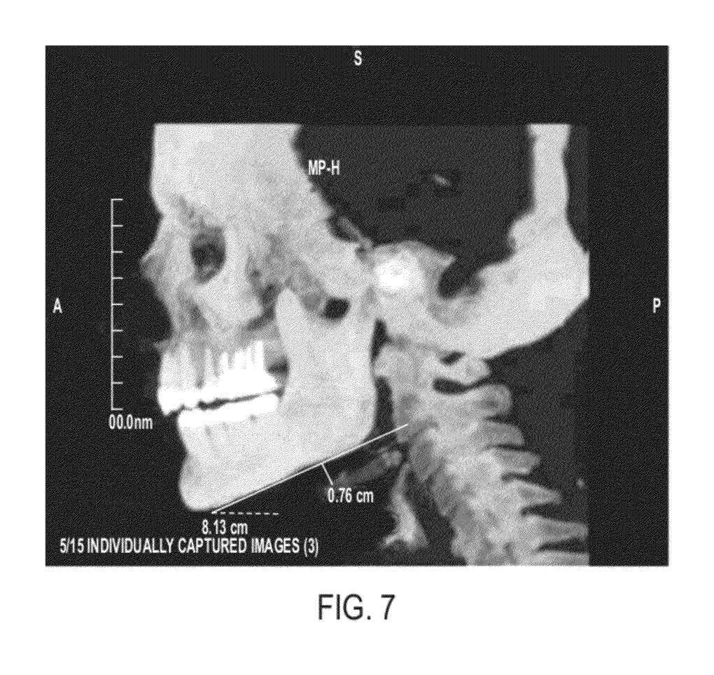

PNS-P is another parameter that represents the distance between PNS 208 and the tip of soft palate, P 216. FIG. 6 illustrates an exemplary PNS-P measurement that has been produced in accordance with the disclosed embodiments. The measured PNS-P in the exemplary depiction of FIG. 6 is 3.86 cm, which is just outside the normal value for this parameter (see Table 1). Another parameter is MP-H, which represents the distance between the mandibular plane (MP) 218 and the point H 214. FIG. 7 illustrates an exemplary MP-H measurement that has been produced in accordance with the disclosed embodiments. The measured MP-H in the exemplary depiction of FIG. 7 is 0.76 cm, which is considerably higher than the normal range for this parameter (see Table 1). Elevated MP-H indicates a diminished distance between the hyoid bone and the back wall of the throat. The posterior tongue inserts on the hyoid bone and thereby, more elevated MP-H numbers indicate narrower airway space at the base of tongue level.

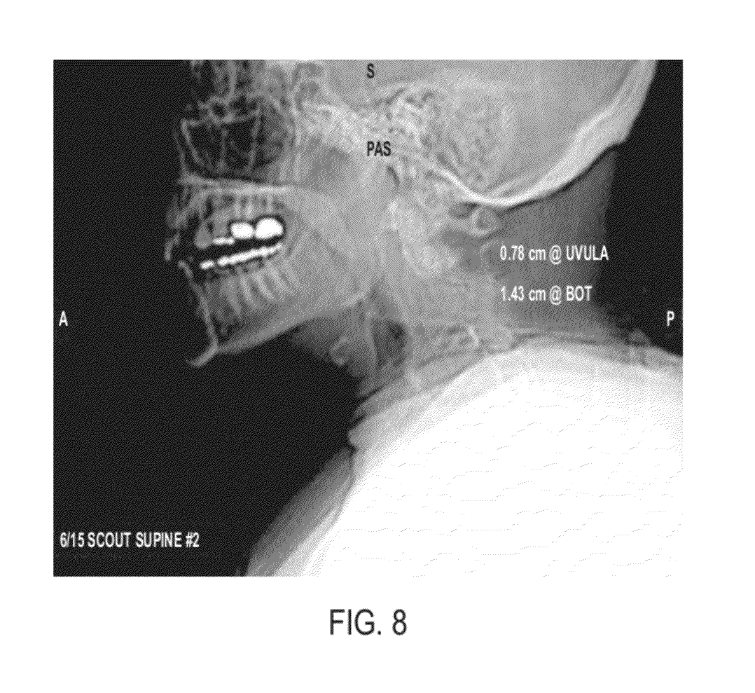

The disclosed parameters further enable precise and accurate one-, two- and/or three-dimensional measurements associated with different sections of the airway. For example, PAS at uvula and PAS at BOT represent one-dimensional (i.e., diagonal) measurements of the posterior air space at uvula and at base of the tongue, respectively. FIG. 8 illustrates exemplary PAS at uvula and PAS at BOT measurements that have been produced in accordance with the disclosed embodiments. The exemplary depiction of FIG. 8 corresponds to a lateral image of the airway and the measured parameters capture the anterior to posterior airway distances. The measured PAS values at uvula and at BOT in the exemplary depiction of FIG. 8 are 0.78 and 1.43 cm, respectively, which are outside the normal range for these parameters (see Table 1). In other examples, one-dimensional measurements of the airway other than at uvula and base of the tongue may be additionally or alternatively conducted. In one example, a distance measurement at the narrowest point of the airway is measured. Determination of the narrowest point of the airway can be conducted by examining the two- and/or three-dimensional images that are produced in accordance with the disclosed embodiments.

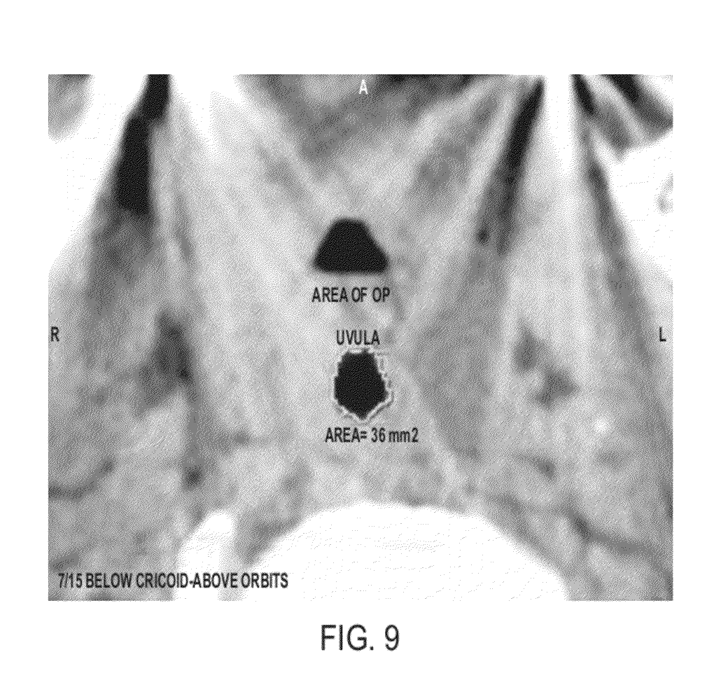

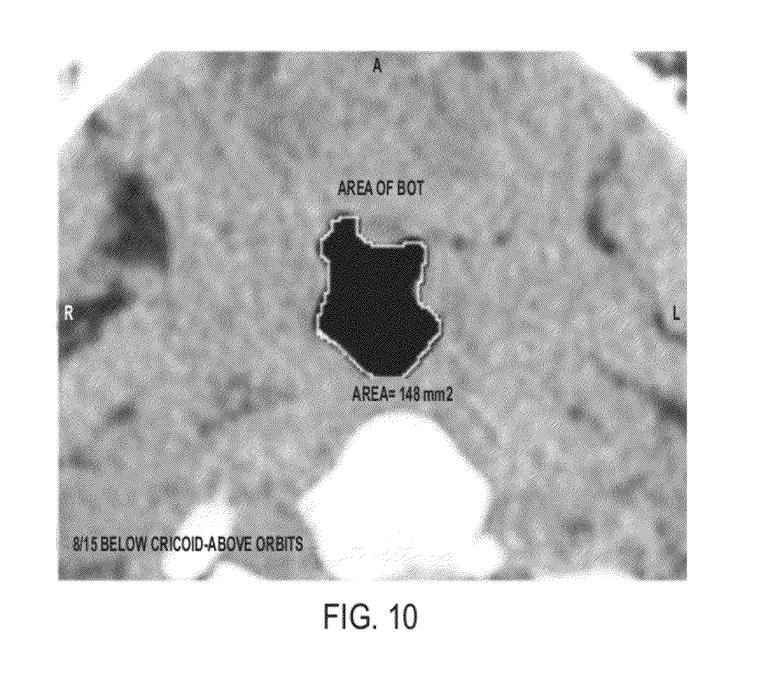

Area of OP at uvula and area of OP at the base of tongue are examples of two-dimensional parameters that can be measured pursuant to the disclosed embodiments. FIG. 9 illustrates an exemplary measurement of the area of OP at uvula that has been produced in accordance with the disclosed embodiments. The measured area of OP at uvula in the exemplary depiction of FIG. 9 is 36 mm², which is considerably lower than the normal range for this parameter (see Table 1). This lower than normal value indicates a narrowing of the airway at the uvula, which can be another contributing factor to the patient's obstructive sleep apnea. FIG. 10 illustrates another exemplary measurement corresponding to the area of OP at BOT that has been produced in accordance with the disclosed embodiments. The measured area of OP at BOT in the exemplary depiction of FIG. 10 is 148 mm², which is within the normal range for this parameter (see Table 1). In other examples, two-dimensional measurements of sections of the airway other than at uvula and base of the tongue may be additionally or alternatively conducted. In one example, an area measurement at the narrowest point of the airway is measured. Determination of the narrowest point of the airway can be conducted by examining the two- and/or three-dimensional images that are produced in accordance with the disclosed embodiments

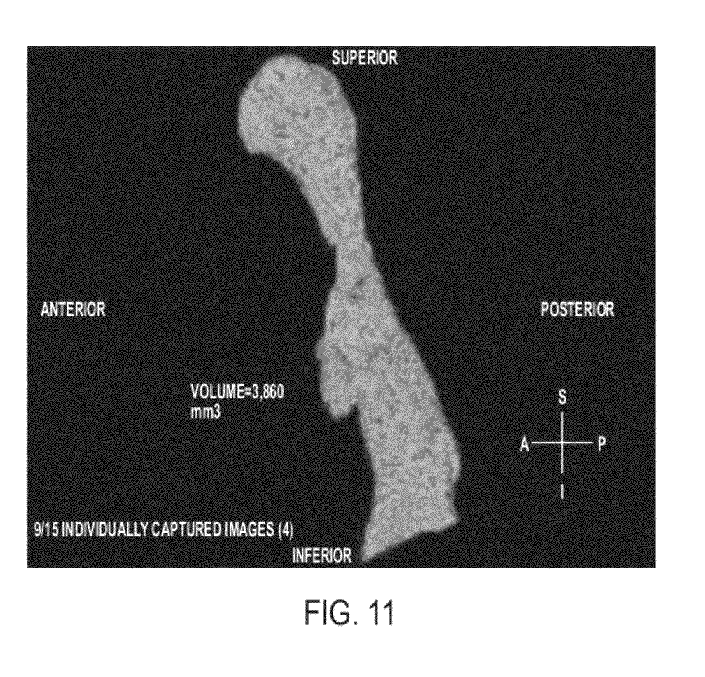

According to the disclosed embodiments, volume of pharynx and/or larynx may also be measured. The three-dimensional measurements facilitate the overall assessment of the airway by producing a value for total capacity of airway. Such measurements may be conducted for the entire volume of the airway. Additionally, or alternatively, such volume measurements may be conducted for only a portion of the airway. In one example, the airway is divided into several overlapping or non-overlapping sections, and the volume of each section is separately measured and reported. In another example, the volume of the entire airway is measured and reported. FIG. 11 illustrates an exemplary volume measurement that has been produced in accordance with the disclosed embodiments. The depicted airway volume in the exemplary illustration of FIG. 11 extends from the back of the nasal airway to the level of the vocal cords. The measured volume in the exemplary depiction of FIG. 11 is 3860 mm³.

In other embodiments, two or more of the one-, two- or three-dimensional measurements and/or images may be combined to collectively and, in some embodiments iteratively, assess the airway characteristics. For example, a three-dimensional image can be used to identify one of more obstructions within the airway. The three-dimensional images may further be rotated in any of the X-, Y-, or Z-directions, or combinations thereof, (assuming Cartesian coordinates) to identify one or more regions of interest. In one embodiment, such regions of interest include sections where an obstruction or narrowing of the airway has occurred. In another example, the region of interest is the narrowest region of the airway. Once such regions of interest are indentified, distance, area and/or or volume measurements within those regions can be conducted. In some embodiments, the one or more regions of interest are manually annotated by a viewer of the images. For example, a user views the electronic images on a display and marks the regions of interest using a mouse, stylus, his/her fingertips (assuming touch screen capabilities are available), and the like. In other examples, the regions of interest are marked on a printout (e.g., paper or film prints) of the images using a pen, pencil, marker and the like. The marked copies of the images are then scanned, or otherwise input, to a computer to allow the appropriate measurements to be conducted. In still other embodiments, the regions of interest are automatically recognized. For example, one or more sections of the airway with unusual characteristics (e.g., sections with the smallest cross-sectional area, smallest diameter, abnormal protrusions, etc.) are automatically detected and the appropriate distance, area or volume measurements are conducted. In some embodiments, the automatic recognition of the regions of interest can be conducted by storing normal or standard values and/or images that are used for comparison with values and/or images associated with the current patient. In other examples, the regions of interest are recognized using neural networks. As also noted earlier in connection with the identification of landmarks in FIG. 1, various image and feature recognition techniques may be used to facilitate the recognition of various landmarks and regions of interest.

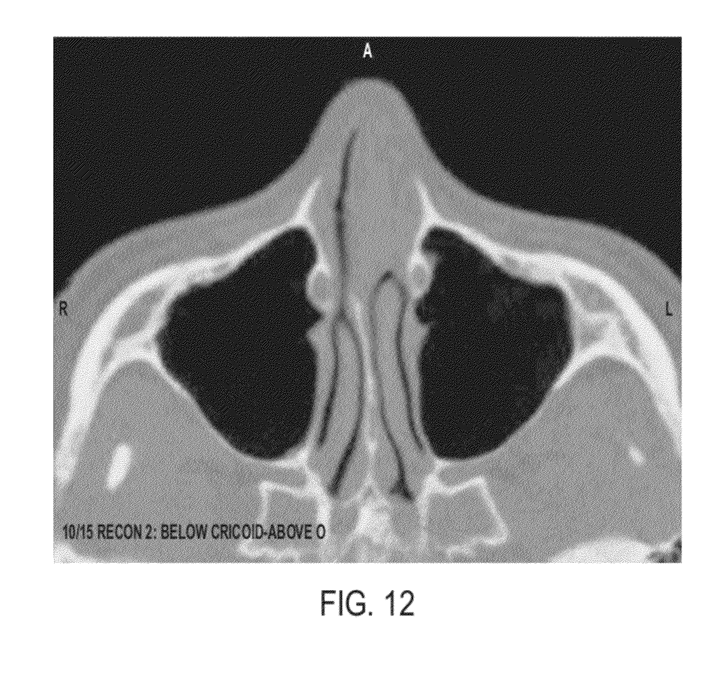

In other embodiments, an assessment of nasal fossa can be conducted to identify septal deviations, nasal spurring, concha bullosa, turbinate hypertrophy, sinusitis, adenoid swelling, inferior turbinate position/swelling, sinusitis, osteomeatal unit occlusion, narrowness of bony nasal structures, prominence of maxillary crest/spine, adenoid swelling, polyps and other anatomic variants associated with nasal fossa narrowing. Similar to the disclosed embodiments related to conducting one-, two- or three-dimensional measurements, the detection of nasal abnormalities can be conducted manually or automatically (or combinations thereof), using the images that are captured (e.g., in step 102 of FIG. 1) or reconstructed (e.g., in step 104 of FIG. 1). FIG. 12 illustrates an exemplary depiction of the nasal cavity that has been produced in accordance with the disclosed embodiments. This axial image indicates that this patient has significant septal deviation, swelling of the anterior nasal mucosa and lining and may have a growth or polyp in the left nasal cavity. The sinus cavities appear clear, without significant sinus infection, the adenoid size is normal, and the bony structures are normal.

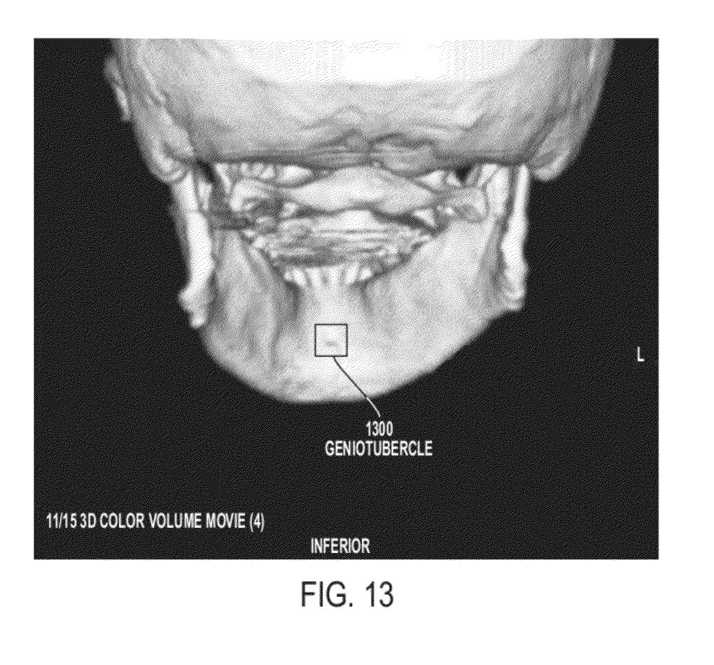

The disclosed embodiments further enable the precise identification of the location of geniotubercle. Geniotubercle is a small part of the jaw bone located on the inner surface of the mandible in the midline directly behind the chin. The genioglossus muscle is suspended from the geniotubercle and the tongue attaches along the whole inner surface of the mandible at the geniotubercle. As noted earlier, in the genioglossus Advancement (GGA) procedure, a small slice is made in the lower jaw and the piece of bone along with the attachment for the tongue is pulled forward and down, then fastened to the outside of the lower jaw. Therefore, the identification of the precise location of the geniotubercle is essential for a successful GGA procedure. According to the disclosed embodiments, the location of the geniotubercle is accurately identified using, for example, surface renderings of the three-dimensional volume associated with the captured images. FIG. 13 illustrates an exemplary location of the geniotubercle 1300 that has been produced in accordance with the disclosed embodiments. The disclosed embodiments enable an accurate and reproducible determination of the specific instertion location of the genioglossus muscle tendon into the bony projection. Such a location can be determined relative to the location of another portion of the jaw and/or teeth. For example, the specific location of geniotubercle can be precisely determined relative to the location of one or more teeth, the location of mandible/madibular plane, the location of one or more dental roots and/or the location of mental foramen (i.e., one of two holes located on the anterior surface of the mandible that permits passage of mental nerve and vessels).

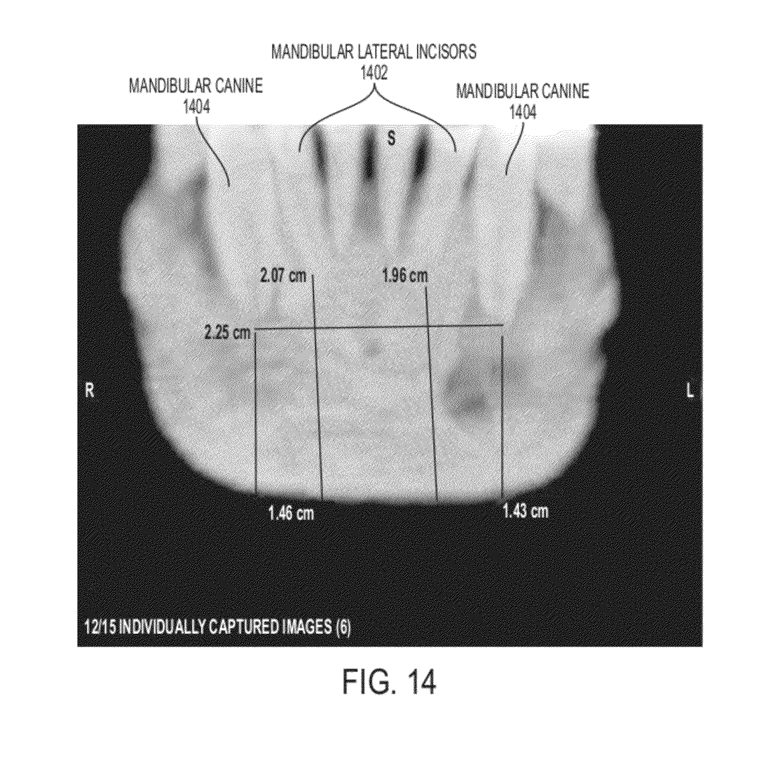

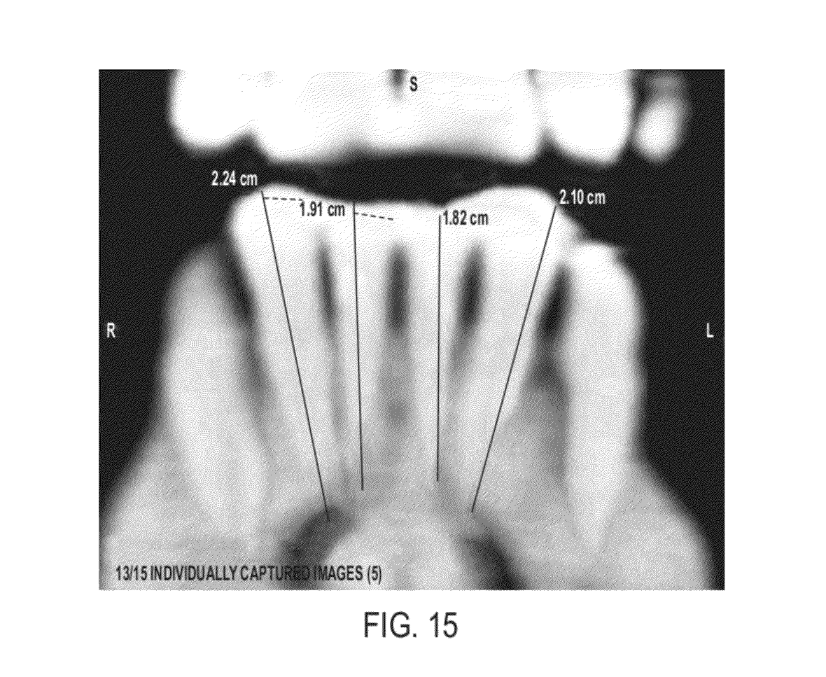

In other embodiments, various characteristics associated with patients' teeth are accurately produced. In particular, distances from the tip of mandibular canines, mandibular lateral incisors, mandibular central incisors or other teeth to the bottom of the mandible are measured. In some embodiments, the distance between the teeth are also measured. These measurements, which enable precise determinations of the locations and distances between various teeth, provide valuable information both for pre-operation planning, as well as for carrying out the actual surgical procedures. FIG. 14 illustrates an exemplary depiction of teeth measurements conducted in accordance with the disclosed embodiments. In particular, FIG. 14 illustrates mandibular canine to bottom of mandible measurements of 1.43 cm and 1.46 cm, mandibular lateral incisor to bottom of mandible measurements of 2.07 cm and 1.96 cm, and a distance between canines of 2.25 cm. FIG. 15 shows another exemplary depiction of teeth measurements that are conducted in accordance with the disclosed embodiments. In particular, FIG. 15 shows the length of roots associated with madibular lateral incisors (i.e., 2.24 cm and 2.10 cm) and mandibular central incisors (i.e., 1.91 cm and 1.82 cm). Determination of the exact locations and the depths of the roots is critical in planning and conducting various surgical procedures, such as the GGA, in a safe manner. In particular, the disclosed embodiments enable precise mapping of the dental roots and, therefore, eliminate and/or drastically reduce inadvertent damage to the roots that could result from imprecise and ad-hoc attempts to estimate the locations and lengths of dental roots. Such imprecise mapping attempts have resulted in numerous malpractice claims, which can be nearly eliminated by utilization of the disclosed techniques

The various measurements that are carried out in accordance with the disclosed embodiments may be conducted manually or automatically, or both. For example, after identification of landmarks, regions of interest, various angular, one-, two- or three-dimensional parameters, a user can manually measure the various distances, angles, cross-sectional areas, volumes and other quantities of interest. Additionally, or alternatively, such measurements may be conducted automatically, for example, by using a computer software. Further, a combination of manual and automatic operations may be conducted. In one example, a user clearly identifies the lines, angles, areas or volumes of interest that are input to a computer. The computer (i.e., a particular software and/or hardware module residing on the computer) then conducts the appropriate measurements to produce the corresponding values.

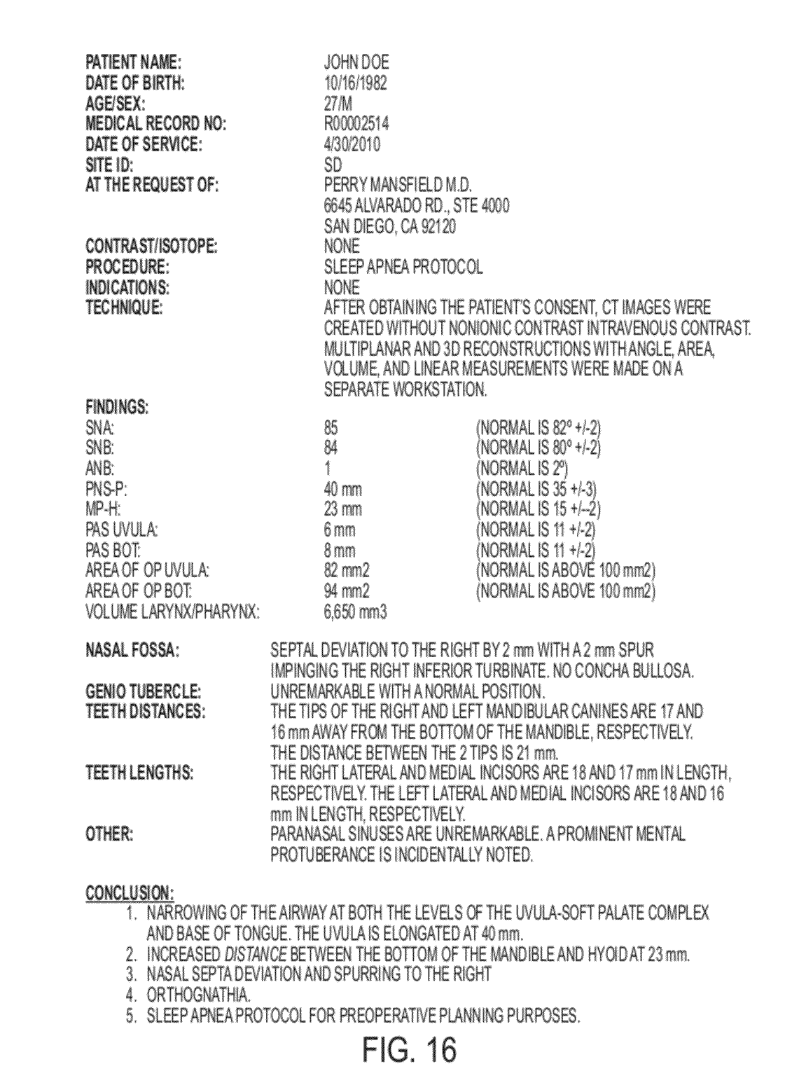

Upon the completion of the measurements a large number of parameters are obtained that allow a complete assessment of the patient's airway. Such an assessment may be accompanied by a report that details a portion or all of the measured parameters. The report may further provide a diagnosis of the various problems associated with the patient's airway. FIG. 16 illustrates an exemplary report that has been generated in accordance with the disclosed embodiments for patient “John Doe.” Under the heading “FINDINGS,” the report in FIG. 16 provides several measured parameters, as well as their normal range of values. In addition, the report provides assessments related to nasal fossa, geniotubercle, teeth distances and teeth lengths. The report also provides “OTHER” findings of interest. In the exemplary report of FIG. 16, the presence of a prominent mental protuberance is listed under the “OTHER” findings. At the end of the report, several conclusions are listed. These conclusions provide an overall assessment of the airway based on the measured parameters and observations made by a radiologist and/or based on automatic recognition techniques. In one example, the conclusions are listed according to their order of importance. For example, the most sever airway obstructions are listed first.

The report may also include a number of images to assist the radiologist and/or surgeon to view the detected landmarks and areas of interest. FIGS. 3-15 provide examples of such images. In addition, the report may also include movies with two- and three-dimensional animation features that allow a user to view a series of different scans, at different rotation angles and speeds. Such images and movies can allow the user to select new areas of interest and conduct additional measurements.

According to the disclosed embodiments, a comprehensive and accurate assessment of a patient's craniofacial features can be conducted based on images that are obtained in a single imaging session (for example, using CT scan). Such images may be used to provide a number of two- and three-dimensional images and videos, as well as a number of measurement values that accurately characterize the various aspects of the patient's airway. In particular, when using images from a CT scan, sagittal and coronal planes can be precisely identified and subsequently used to identify the locations of various landmarks with high accuracy. As a result, the various craniofacial measurements (e.g., SNA, SNB, ANB, etc.) are produced with high levels of confidence. The produced measurements correspond to different angles, distances, areas and volumes associated with landmarks and areas of interest. Comparison of such measurements with standard or normal values can positively identify any and all sources of blockage in the patient's airway. Such a comprehensive assessment allows a physician to identify the proper surgical and, perhaps non-surgical, procedures for treating sleep apnea. For example, the comprehensive assessment that is conducted in accordance with the disclosed embodiments can identify multiple sources of blockage that must be treated with several surgical procedures. The comprehensive assessment that is conducted in accordance with the disclosed embodiments further identifies the extent of each blockage and can, therefore, provide a guide as to the priority of required treatment.

Another aspect of the disclosed embodiments relates to reproducibility and redundancy of measured parameters. Since the images (e.g., CT scans) are available to the user (e.g., radiologist, surgeon, etc.), they can be used to repeat certain measurements at any time after the initial imaging session. For example, the user can manually verify the accuracy of automated landmark detections and/or automated parameter measurements. In addition, the user can make additional measurements at the vicinity of an originally identified area of interest. Such additional measurements provide assurances regarding the validity of the original measurements. Furthermore, since a large number of parameters can be produced (e.g., cross-sectional area measurements at different levels, volume measurements at different subsections of the airway, various angular measurements, etc.), the comprehensive assessment that is conducted in accordance with the disclosed embodiments comprises a considerable amount of redundancy. Such redundant information and measurements can be collectively analyzed to identify the source and extent of airway blockage with high levels of accuracy. The above noted features of the disclosed embodiments, therefore, produce extremely high success rates for identification and diagnosis of anatomic upper airway narrowing which results in sleep apnea and/or other sleep disorders.

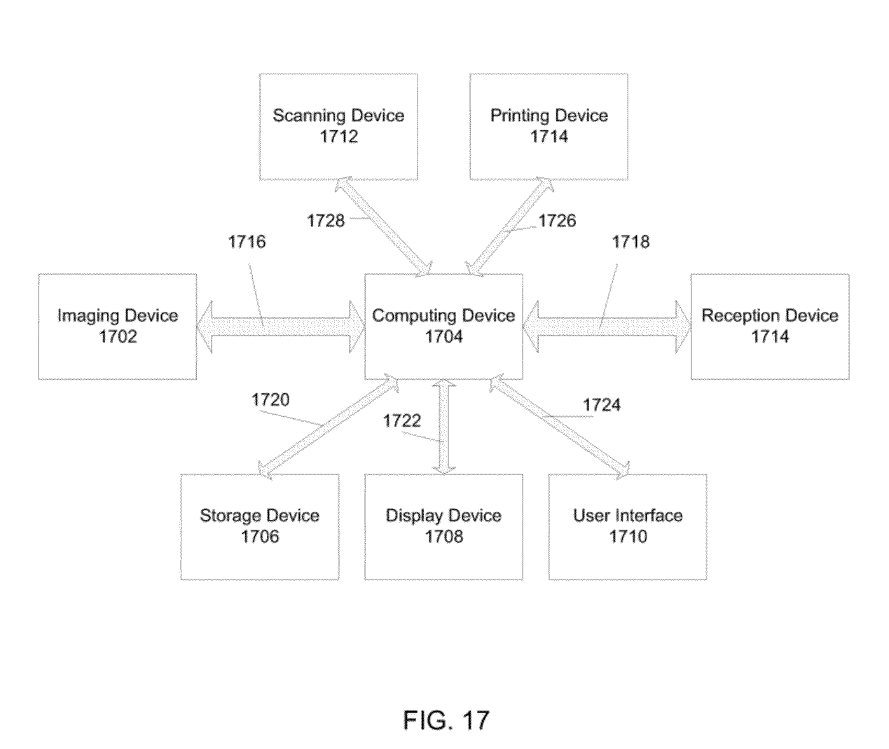

FIG. 17 is a diagram that illustrates various components within an exemplary system in accordance with the disclosed embodiments. The image scanning device 1702 is responsible for acquiring craniofacial images, such as X-Ray, CT scan, MRI, ultrasound and other devices. The images are communicated to a computing device 1704 through a communication link 1716. The communication link 1716 can be any one of a wired or wireless communication links and can utilize a variety of link communication protocols. The images that are obtained from the scanning device 1702 may also be communicated through the Internet to the computing device 1704 and/or delivered to the communication device 1704 through a storage medium, such as a CD, DVD, Flash memory device, and the like. The computing device 1704 is responsible for receiving the images and conducting various processing operations that were described throughout this disclosure. By the way of example, and not by limitation, these operations include rendering two- and three-dimensional images, performing image and feature recognition, making measurements associated with craniofacial parameters, creating a report and the like.

The computing device 1704 may also be in communication with a storage device 1706, a display device 1708 and a user interface 1710. The storage device may comprise data or program code that is accessed by the computing device 1704 and/or are used to configure the computing device 1704 to conduct various operations. A user can control and/or interact with the computing device through the user interface 1710. Such interactions may include marking certain portions of images, entering various commands and data, printing, scanning and the like. The display 1708 enables the user to monitor the operation of the computing device 1704 and to view various images at different stages of processing. If equipped with touch screen capabilities, the display 1708 may also allow the user to enter commands, make selections, mark images and conduct other operations. The storage device 1706, the display device 1708 and the user interface 1710 may be integrated as part of the computing device 1704, or may be communicatively connected to the computing device 1704 through one or more communication links 1720, 1722 and 1724. The communication links 1720, 1722 and 1724 can be any one of a wired or wireless communication links and can utilize a variety of link communication protocols.

The computing device 1704 may be connected to a scanning device 1712 and a printing device 1714. The scanning device 1712 allows scanning of images that are, for example, modified by a user to include identification marks associated with certain landmarks and areas of interest. The printing device 1714 allows reports, images and other files to be printed during different stages of processing. The scanning device 1712 and the printing device 1714 are communicatively connected to the computing device 1704 through one or more communication links 1728 and 1726, respectively. The communication links 1726 and 1728 can be any one of a wired or wireless communication links and can utilize a variety of link communication protocols.

In the exemplary system that is depicted in FIG. 17, a reception device 1714 is also communicatively connected to the computing device 1704 through the communication link 1718. The communication link 1718 can be any one of a wired or wireless communication links and can utilize a variety of link communication protocols. The communication link 1718 can additionally or alternatively include a storage medium, such as a CD, DVD, Flash memory device, and the like, that is used to communicate various reports, images and videos to the reception device 1714. A user, such as a surgeon, can use the reception device to view, print or manipulate the received reports, images, videos and other data.

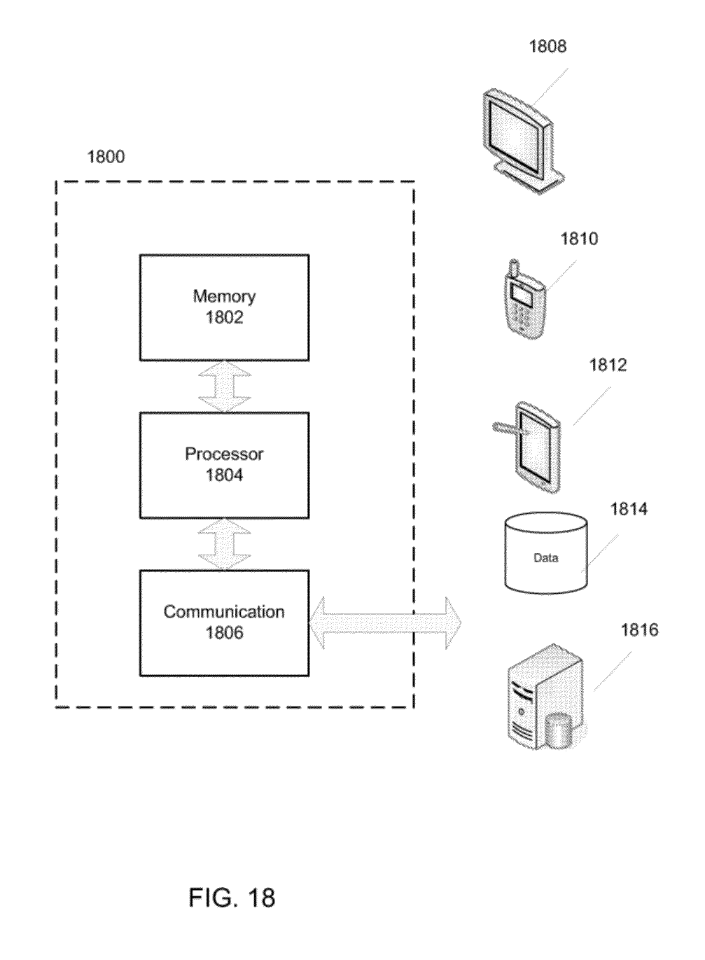

It is understood that the various embodiments of the present invention may be implemented individually, or collectively, in devices comprised of various hardware and/or software modules and components. These devices, for example, may comprise a processor, a memory unit, an interface that are communicatively connected to each other, and may range from desktop and/or laptop computers, to consumer electronic devices such as media players, mobile devices and the like. For example, FIG. 18 illustrates a block diagram of a device 1800 within which all or portions of the various embodiments may be implemented. The device 1800 can, for example, represent the computing device 1704 or the reception device 1714 that are depicted in FIG. 17. The device 1800 comprises at least one processor 1802 and/or controller, at least one memory 1804 unit that is in communication with the processor 1802, and at least one communication unit 1806 that enables the exchange of data and information, directly or indirectly, with other entities, devices and networks 1808 to 1816. For example, the communication unit may be communicating with a display 1808, a mobile phone 1810, a mobile computer 1812 (e.g., a laptop computer, an ipad, an ipod, a table PC, etc.), a database 1814 and/or a server 1816. The communication unit 1806 may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. The device 1800 that is depicted in FIG. 18 may reside as a separate component within or outside a larger device.

Similarly, the various components or sub-components within each module of the present invention may be implemented in software, hardware, firmware. The connectivity between the modules and/or components within the modules may be provided using any one of the connectivity methods and media that is known in the art, including, but not limited to, communications over the Internet, wired, or wireless networks using the appropriate protocols.

Various embodiments described herein are described in the general context of methods or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the disclosed embodiments can be implemented as computer program products that reside on a non-transitory computer-readable medium. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. The features of the embodiments described herein may be combined in all possible combinations of methods, apparatus, modules, systems, and computer program products.

What is claimed is:

1. A method, comprising:
processing a plurality of images associated with craniofacial features of a patient, the images having been produced using computed tomography (CT);
identifying a plurality of landmarks within the processed images;
conducting a plurality of measurements in accordance with the identified landmarks to produce one or more parameters; and
producing a comprehensive assessment of the patient's airway in accordance with the parameters.

2. The method of claim 1, wherein the comprehensive assessment is produced by comparing the measured parameters to a set of normal values.

3. The method of claim 1, further comprising:
identifying one or more characteristics of the airway using the processed images; and
producing the comprehensive assessment in accordance with the identified characteristics.

4. The method of claim 3, wherein:
the characteristic is a volume and shape of the airway; and
the comprehensive assessment comprises a volume measurement associated with one or more sections of the airway.

6. The method of claim 4, wherein the one or more sections of the airway are selected from a group of sections of the airway consisting of:
a section of the airway with a narrowing;
a section of the airway at uvula at its narrowest airway point in a lateral dimension; and
a section of the airway at base of tongue at its narrowest point in a lateral projection.

7. The method of claim 3, wherein:
the identified characteristic is an area of the airway;
and the comprehensive assessment comprises an area measurement associated with one or more sections of the airway.

8. The method of claim 7, wherein the one or more sections of the airway are selected from a group of sections of the airway consisting of:
a section of the airway with a smallest cross-sectional area;
a section of the airway with a narrowing;
a section of the airway at uvula; and
a section of the airway at base of tongue.

9. The method of claim 3, wherein
the identified characteristic is associated with nasal fossa; and
the comprehensive assessment comprises identified abnormalities within the nasal fossa.

10. The method of claim 9, wherein the identified abnormalities are selected from a group of abnormalities consisting of:
a septal deviation;
a nasal spurring;
concha bullosa;
inferior turbinate position/swelling;
sinusitis;
osteomeatal unit occlusion;
narrowness of bony nasal structures; prominence of maxillary crest/spine;
adenoid swelling; and
polyps.

11. The method of claim 1, wherein a plurality of abnormalities within the airway are identified.

12. The method of claim 1, further comprising identifying a specific location of geniotubercle relative to another location selected from a group consisting of:
a tooth;
mandible;
a dental root; and
a mental foramen.

13. The method of claim 1, further comprising identifying a distance between teeth or a length of a tooth.

14. The method of claim 1, wherein the plurality of images are produced in a single imaging session.

15. The method of claim 1, wherein the processing comprises rendering two- and three-dimensional images of the airway.

16. The method of claim 1, wherein the parameters are selected from a group of parameters consisting of:
an SNA angle;
an SNB angle;
an ANB angle;
a PNS-P distance;
an MP-H distance;
a posterior airway space (PAS) at uvula distance; and
a PAS at base of tongue (BOT) distance.

17. The method of claim 1, wherein the landmarks are automatically identified using a computer-implementable algorithm.

18. The method of claim 1, wherein the landmarks are identified by manually viewing the images.

19. The method of claim 1, wherein the measurements are conducted automatically using a computer-implementable algorithm.

20. The method of claim 1, further comprising producing a report comprising a description of one or more abnormalities of the patient's airway.

21. The method of claim 1, wherein the produced assessment provides a reliable diagnosis of sleep apnea.

22. A device, comprising:
a processor; and
a memory comprising processor executable code, the processor executable code, when executed by the processor, configures the device to:
process a plurality of images associated with craniofacial features of a patient, the images having been produced using computed tomography (CT);
identify a plurality of landmarks within the processed images;
conduct a plurality of measurements in accordance with the identified landmarks to produce one or more parameters; and
produce a comprehensive assessment of the patient's airway in accordance with the parameters.

23. A computer program product, embodied on a computer-readable medium, comprising:
computer code for processing a plurality of images associated with craniofacial features of a patient, the images having been produced using computed tomography (CT);
computer code for identifying a plurality of landmarks within the processed images;
computer code for conducting a plurality of measurements in accordance with the identified landmarks to produce one or more parameters; and
computer code for producing a comprehensive assessment of the patient's airway in accordance with the parameters.
